(19) United States
(12) Patent Application Publication
(10) Pub. No.: US 2020/0246233 A1
(43) Pub. Date: Aug. 6, 2020

(54) ORAL CARE PRODUCT

(71) Applicant: Belle-Aire Fragrances, Inc., Mundelein, IL (US)

(72) Inventor: Donald Conover, Mundelein, IL (US)

(21) Appl. No.: 16/265,680

(22) Filed: Feb. 1, 2019

(51) Int. Cl. A61K 8/27; A61Q 11/00

(52) U.S. Cl. CPC A61K 8/27; B82Y 5/00; A61Q 11/00

(57) ABSTRACT

A product comprising a zinc oxide nanoparticle onto which one or more natural odor neutralizers are adsorbed. The product can be used as a component of an oral care product such as a mouthwash, a mouth rinse, a toothpaste, a mint, a chewing gum, or as a component of a personal care product.

### FIELD OF THE INVENTION

This invention is directed to a method to control malodors, in particular malodors from a human, more particularly from a human mouth. The invention includes a natural odor neutralizer.

### BACKGROUND OF THE INVENTION

Oral malodor, also referred to as mouth odor, halitosis, or bad breath, is a common problem which has many possible causes and for which many solutions have been proposed. Products used to address bad breath include toothpastes, mouth washes and mouth rinses, tongue scrapers, chewing gum, breath mints, breath sprays, as well as home-made solutions such as baking soda, hydrogen peroxide, leafy plants such as parsley, and other items and materials. Although solutions can lie in improved dental care and avoidance of certain foods, many mouth odor products merely mask the malodor, and do not eliminate it.

Oral malodor is formed by gram negative bacteria in the mouth. Accordingly, one approach to reduce oral malodor is to combat these bacteria with classical antibacterial agents such as triclosan cetyl-pyridinium chloride and chlorhexidine. These agents, while suited for mouthwash, are not practical for other applications such as breath mints and chewing gum.

Another approach is enzymatic inhibition of the relevant bacterial enzymes so that the malodorous sulfur volatiles are not formed in the first place. This approach has been successfully applied to the enzymes that produce methyl mercaptan, the volatile sulfur compound that is a key contributor to morning breath, and halitosis. This approach, however, does not work for other types of bad breath such as garlic breath, onion breath, and coffee breath.

It is beneficial to address bad breath with a product that neutralizes the odor rather than merely masking the odor. Products are known that help neutralize malodors in the mouth, such as U.S. Pat. No. 9,408,810 B2, the subject matter of which is incorporated herein by reference. Some of the known products, however, tend to be washed out of the mouth, either by rinsing or by natural salivation. A need exists for an oral care product that provides time-release properties.

Additionally, the impression of fresh and clean is critical for oral care products. After all, there is not much point in using a product that leaves the mouth feeling unclean. While flavors such as mint have a relatively short-term effect of masking oral malodors, they do not, however, necessarily eliminate them.

The use of zinc oxide in microparticles is known for treating unpleasant odors, such as bad breath and body odor. For example, in WO 99/59539, Odour Absorbing Agent, zinc oxide particles having a particle size as low as 10 nanometers (nm) is used to absorb the molecules that cause bad breath and body odor.

The use of zinc oxide for time release of pharmaceutical agents has been described in, for example, U.S. Pat. No. 6,399,103 to Yutaka, Method of producing a sustained-release preparation.

The present invention attempts to meet the inadequacies of the prior art and the long-felt need for oral malodor reduction.

### SUMMARY OF THE INVENTION

The preferred embodiment of the method of the present invention comprises a natural odor neutralizer adsorbed onto zinc oxide nanoparticles.

### A BRIEF DESCRIPTION OF THE DRAWINGS

The organization and manner of the structure and operation of the invention, together with further objects and advantages thereof, may best be understood by reference to the following description, taken in connection with the accompanying non-scale drawings, including charts and tables and, where appropriate, like reference numerals identify like elements.

FIG. 1 is a table listing the molecules responsible for bad breath.

FIG. 2 is a table listing the types of molecules responsible for unpleasant body and environmental odors.

FIG. 3 is a flow chart of the protocol for testing of the inventive product in breath mints and chewing gum.

FIG. 4 is a table listing the dilution series for garlic odor standard solutions.

FIG. 5 is a chart of the garlic odor intensity scale used for testing the inventive product in breath mints and chewing gum.

FIG. 6 is a table of sampling times for testing the inventive product in breath mints and chewing gum.

FIG. 7 is a table summarizing the results of testing the inventive product in breath mints.

FIG. 8 is a chart of the effect of the inventive products in breath mints.

FIG. 9 is a chart of the effect size of oral garlic intensity reduction due to the addition of the inventive product to breath mints.

FIG. 10 is a table is a table summarizing the results of testing the inventive product in chewing gum.

FIG. 11 is a chart of the effect of the inventive products in chewing gum.

FIG. 12 is a chart of the effect size of oral garlic intensity reduction due to the addition of the inventive product to chewing gum.

FIG. 13 is a table of gas chromatography peak areas for SPME analysis of ally methyl sulfide and diallyl disulfide.

FIG. 14 is a chart of the effective of the inventive product on the headspace concentration of ally methyl sulfide and diallyl disulfide.

FIG. 15 is a chart of the effect of the inventive product in breath mints and in chewing gum on oral garlic odor intensity.

FIG. 16 is a flow chart of the protocol for testing of the inventive product in toothpaste.

FIG. 17 is a table of sampling times for testing the inventive product in toothpaste.

FIG. 18 is a chart of the effect of the inventive product in toothpaste on oral garlic odor intensity.

FIG. 19 is a table summarizing the results of the effect of the inventive product in toothpaste on oral garlic odor intensity.

FIG. 20 is a chart of the effect size of oral garlic odor intensity reduction due to the use of the inventive product in toothpaste.

FIG. 21 is a table of gas chromatography peak areas for SPME analysis of ally methyl sulfide and diallyl disulfide.

FIG. 22 is a chart of the effect of the inventive product on the headspace concentration of allyl sulfide and diallyl disulfide.

FIG. 23 is a diagrammatic representation of covalent bond formation between the molecules of the inventive product and malodor molecules.

FIG. 24 is a diagrammatic representation of the magnetic-like and Van der Waals forces of attraction between the molecules of the inventive product and malodor molecules.

FIG. 25 is a diagrammatic representation of mixed micelle formation by the molecules of the inventive product, malodor molecules, and water.

### DETAILED DESCRIPTION OF THE EMBODIMENTS

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein. The invention will be described as used in an oral care product for humans. The invention can also be used in an oral care product for other animals. The invention can also be used in deodorant products for humans or other animals. The invention can also be used as a general odor reducing agent.

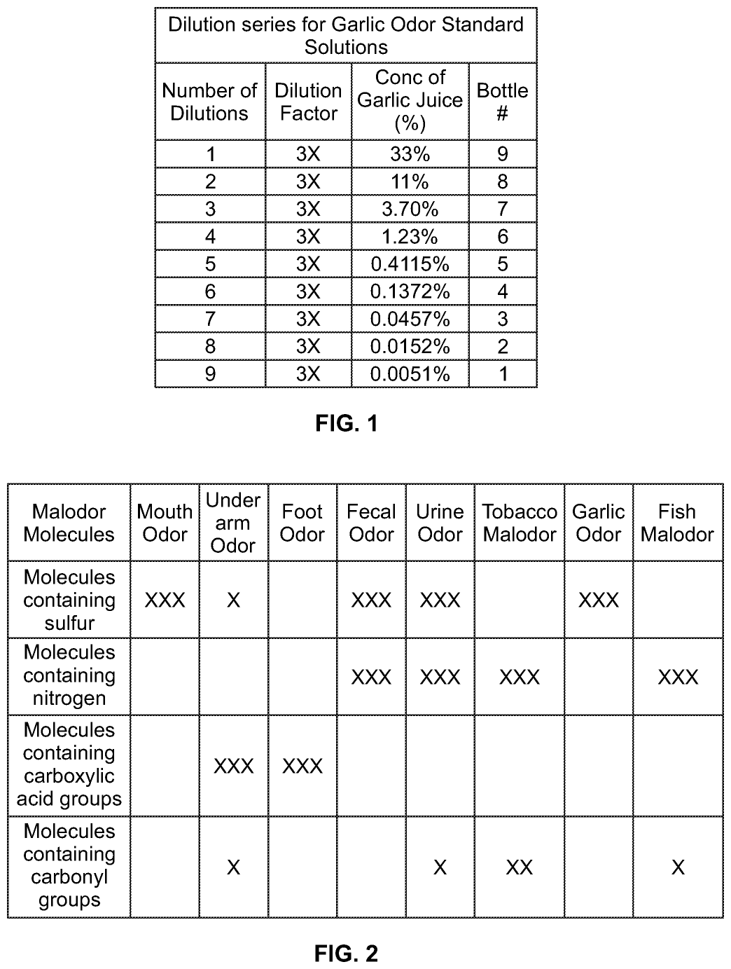

Nonpathological halitosis, which is generally referred to as physiological halitosis or endogenous breath malodor, is due to bacteria in the oral cavity converting food particles in the oral cavity into volatile sulfur compounds. These volatile sulfur compounds, primarily hydrogen sulfide and methyl mercaptan, can lead to an oral malodor that is beyond socially acceptable levels. Other causes of bad breath are garlic and onion which also generate malodorous volatile sulfur compounds in the oral cavity. These bad breath molecules are illustrated in FIG. 1.

Other causes of halitosis, sometimes called exogenous breath malodor, are from ingestion of foods and beverages containing malodorous volatile sulfur compounds. The foods and or beverages particularly notorious for generation of bad breath are garlic, onion, and coffee. These volatile sulfur compounds are also illustrated in FIG. 1.

Garlic was chosen as the bad breath generator for the testing of the inventive products because of garlic's commercial availability in crushed and juice form and because it generated volatile sulfur compounds that were similar in structure to those responsible for morning breath and halitosis. Garlic breath was chosen as a good model for all types of bad breath since garlic odor is strong and very unpleasant. The volatile sulfur compounds responsible for garlic breath are representative of the volatile sulfur compounds responsible for most other types of bad breath. A product that will control garlic breath will control most other causes of oral malodor.

It was, in fact, found by solid phase micro-extraction SPME headspace analysis that the inventive products that formed molecular complexes with the garlic volatiles also formed molecular complexes with methyl mercaptan and dimethyl sulfide.

In order to show both the immediate and sustained malodor counteraction ability of the inventive products, a garlic meal was designed that generated an immediate very strong garlic odor that persisted for at least three hours. This garlic meal, which is described in detail below, consisted of eating crushed garlic followed by rinsing the mouth with cold pressed garlic juice.

Two primary reasons people purchase gum, mints, and breath fresheners are to freshen their breath and to remove bad taste from their mouths. A fresh breath is one that is devoid of the malodorous molecules responsible for morning breath, halitosis, garlic breath, onion breath, etc. The malodorous molecules are primarily volatile sulfur compounds: hydrogen sulfide, methyl mercaptan, and dimethyl sulfide for morning breath and halitosis; allyl methyl sulfide, and diallyl disulfide for garlic breath; and propanethiol, for onion breath. The structures of these molecules are presented in FIG. 1.

The natural odor neutralizers of the preferred embodiments of the present invention form molecular complexes with all types of sulfur compounds such as, for example, methyl mercaptan, dimethyl sulfide, allyl methyl sulfide, and diallyl disulfide. These molecules, once complexed, are no longer volatile, and cannot reach the nose and be smelled.

With few exceptions, malodor molecules are water-soluble, have relatively low molecular weights, and are hydrogen-bond donors or acceptors. A large subset of malodors can also enter nucleophilic addition reactions with molecules containing reactive double bonds. With few exceptions, malodor molecules contain either a sulfur atom, a nitrogen atom, a carboxylic acid group, a carbonyl group or an alcohol group or combinations of these groups as illustrated in FIG. 2. The molecules making up each malodor class are:
Mouth Odor: hydrogen sulfide, methyl mercaptan, dimethyl sulfide.
Underarm Odor: 3-methyl-2-hexenoic acid, 3-methyl-3-sulfanylhexan-1-ol.
Foot Odor: isovaleric acid.
Fecal odor: indole, skatole, hydrogen sulfide, dimethyl sulfide.
Urine Odor: hydrogen sulfide, methyl mercaptan, dimethylamine, trimethyl amine.
Tobacco Malodor: pyridine, pyrrole, toluene, 3-vinyl pyridine. Over 1000 volatile molecules have been identified in tobacco smoke. The ones presented above are the high concentration components of tobacco smoke that have an unpleasant odor.
Garlic Odor: allyl methyl sulfide. Garlic odor contains a number of volatile sulfur compounds. Allyl methyl sulfide, however, is considered the key contributor to garlic odor.
Fish malodor: dimethylamine, trimethyl amine (these odors are associated with fish that is not fresh).

Since the molecular complexes formed between the natural odor neutralizers of the preferred embodiments of the present invention and malodors rely on the molecular properties of the malodor and not on its smell, a molecule that complexes with dimethyl sulfide will also complex other sulfides such as those present in garlic odor. This result is because complex formation depends upon an electronic property of the sulfur atom and not on the smell of the molecule containing the sulfur atom.

The natural odor neutralizers of the preferred embodiments of the present invention are adsorbed onto zinc oxide nanoparticles to facilitate the sustained release of the natural odor neutralizers. The product comprising the adsorbent zinc oxide nanoparticles and the adsorbate natural odor neutralizers are preferably incorporated into an oral care product so that as a person uses the oral care product, the odor neutralizing agent has an opportunity to eliminate or neutralize malodors. The oral care product may be a product kept in the mouth for a short period, such a mouth rinse, mouth wash, or toothpaste. The oral care product may be one that is kept in the mouth for a longer time, such as lozenges, breath mints, breath-freshening wafers, chewing gum, and the like. The adsorbed nanoparticles can be incorporated into other products as well.

The zinc oxide nanoparticles of the preferred embodiments are preferably manufactured by nanoprecipitation, emulsion-diffusion, or solvent evaporation. The zinc oxide nanoparticles preferably comprise about 85 percent by weight of each adsorbed nanoparticle, with the adsorbate comprising about 15 percent by weight of each adsorbed nanoparticles. This weight ratio can vary by about plus or minus 10 percent.

Nanoscale materials are defined in the United States and in Europe as particulates with at least one external dimension less than 100 nm or with an SSA less than 60 m²/g. In the United States, however, a material with an external dimension as high as 1,000 nm can be considered to have nanoscale-like properties. Accordingly, the zinc oxide nanoparticles of the preferred embodiments of the present invention are not necessarily strictly nanoparticles, but do have nanoscale-like properties.

In the preferred embodiment, the zinc oxide is USP grade, is in excess of 99 percent pure, has a mean particle size of less than 200 nm, with zero percent below 100 nm, and has a specific surface area (SSA) of four to nine square meters per gram (m²/g). Suitable product is supplied by Nanophase Technologies Corp., Romeoville, Ill.

In other embodiments, zinc oxide nanoparticles having a mean particle size of less than 100 nm or zinc oxide nanoparticles up to 1,000 nm, can be used.

The natural odor neutralizer is preferably a combination of natural ingredients that neutralize the chemicals that cause malodor. The natural odor neutralizer of the preferred embodiment comprises one or more of the following natural products: balsam copaiba, bees wax absolute, cardamon essence, hydroxycitronellal, ionone alpha, ionone beta, carvacrol, jasmine absolute, lavender absolute, mimosa absolute, neroli absolute, orange oil, orris absolute, osmanthus absolute, vanilla absolute, violet leaves absolute, davana oil, dimethyl anthranilate, elderflower oil, raspberry ketone, and natural ethyl alcohol.

Preferably, the natural products are in a diluent, preferably a natural. The preferred diluent is triethyl citrate natural, a natural solvent. Similar diluents are acceptable.

The natural odor neutralizer of another embodiment consists essentially of balsam copaiba, bees wax absolute, cardamon essence, hydroxycitronellal, ionone alpha, ionone beta, carvacrol, jasmine absolute, lavender absolute, mimosa absolute, neroli absolute, orange oil, orris absolute, osmanthus absolute, vanilla absolute, violet leaves absolute, davana oil, dimethyl anthranilate, elderflower oil, raspberry ketone, and natural ethyl alcohol.

The natural odor neutralizer of yet another embodiment comprises one or more of the following natural products in the following amounts (in weight percentage):

| | |
|---|---|
| Balsam Copaiba | 0.00020%-5.00000% |
| Bees Wax Absolute | 0.00001%-5.00000% |
| Cardamon Essence | 0.00001%-5.00000% |
| Hydroxycitronellal | 0.05000%-8.00000% |
| Ionone Alpha | 0.05000%-8.00000% |
| Ionone Beta | 0.05000%-7.00000% |
| Carvacrol | 0.05000%-5.00000% |
| Jasmine Absolute | 0.00200%-1.00000% |
| Lavender Absolute | 0.00020%-1.00000% |
| Mimosa Absolute | 0.00001%-1.00000% |
| Neroli Absolute | 0.00001%-1.00000% |
| Orange Oil | 0.00001%-5.00000% |
| Orris Absolute | 0.00001%-0.30000% |
| Osmanthus Absolute | 0.00001%-0.30000% |
| Vanilla Absolute | 0.00001%-1.00000% |
| Violet Leaves Absolute | 0.00001%-0.40000% |
| Davana Oil | 0.00001%-1.00000% |
| Dimethyl Anthranilate | 0.00001%-2.00000% |
| Elderflower Oil | 0.00001%-0.45000% |
| Raspberry Ketone | 0.00001%-5.00000% |
| Natural ethyl alcohol | 0.25%-6% |

The natural odor neutralizer of yet another embodiment consists essentially of the following natural products in the following amounts (in weight percentage):

| | |
|---|---|
| Balsam Copaiba | 0.00020%-5.00000% |
| Bees Wax Absolute | 0.00001%-5.00000% |
| Cardamon Essence | 0.00001%-5.00000% |
| Hydroxycitronellal | 0.05000%-8.00000% |
| Ionone Alpha | 0.05000%-8.00000% |
| Ionone Beta | 0.05000%-7.00000% |
| Carvacrol | 0.05000%-5.00000% |
| Jasmine Absolute | 0.00200%-1.00000% |
| Lavender Absolute | 0.00020%-1.00000% |
| Mimosa Absolute | 0.00001%-1.00000% |
| Neroli Absolute | 0.00001%-1.00000% |
| Orange Oil | 0.00001%-5.00000% |
| Orris Absolute | 0.00001%-0.30000% |
| Osmanthus Absolute | 0.00001%-0.30000% |
| Vanilla Absolute | 0.00001%-1.00000% |
| Violet Leaves Absolute | 0.00001%-0.40000% |
| Davana Oil | 0.00001%-1.00000% |
| Dimethyl Anthranilate | 0.00001%-2.00000% |
| Elderflower Oil | 0.00001%-0.45000% |
| Raspberry Ketone | 0.00001%-5.00000% |
| Natural ethyl alcohol | 0.25%-6% |

Formulations of natural odor neutralizers as described in the embodiments have been branded as Total Malodor ^{Management}® natural odor neutralizers or TMM® natural odor neutralizers, available from Belle-Aire Creations, Mundelein, Illinois, USA. For purposes of this application, the term TMM® means natural odor neutralizers as described in the preceding paragraphs. Also for purposes of this application, the term TMM® NANO means the inventive product described in this application, comprising the adsorbate natural odor neutralizers and the absorbent zinc oxide nanoparticles.

Other natural odor neutralizers are acceptable as well. For example, an acceptable natural odor neutralizer is described in U.S. Pat. No. 9,408,810 B2, referenced above. Another acceptable natural odor neutralizer is All Natural OMC 85946 Finished Flavor Ingredient, available from Belle-Aire Creations, Mundelein, Ill., USA.

The nanoscale properties of the products of the present invention are useful, as at least some of the adsorbed nanoparticles, when used in toothpastes, mouth washes, mouth rinses, breath mints, chewing gum, or other oral care products, will remain in the mouth following ejection from the mouth and/or rinsing. The nanoparticles will become entrained in the saliva and will remain under the gums, between the tissues of the mouth and the teeth. Zinc oxide is only slightly soluble in water, slightly more so under heat, such as the higher temperature in vivo as compared to room temperature. Accordingly, the nanoparticles will degrade slowly in the saliva and under the gums. Since the nanoparticles will have a range of sizes, they will degrade over a period of time, not all at once.

Besides oral care applications, the products of the present invention are useful in other applications to neutralize odor. For example, TMM® NANO can be incorporated into personal care products such as deodorant, whether spray, roll-on, or stick. The adsorbed nanoparticles will adhere in cracks in the skin and to hair, for example, and as they degrade in perspiration, the work as described above to neutralize malodors over a period of time. The product can be used in any application in which time-release properties are needed for a natural odor neutralizer.

BREATH MINTS OR CHEWING GUM

The addition of the product of the preferred embodiments of the present invention to breath mints or chewing gum at levels of 0.1 percent effectively eliminated volatile sulfur compounds associated with halitosis. Belle Aire's sensory science group assessed oral garlic odor that had been induced in the oral cavity of the subjects by their ingesting crushed garlic followed by swishing and expectorating neat, cold-pressed garlic juice. The size of this oral garlic odor intensity reduction was such that any user of the product would perceive the effect.

Belle Aire's analytical team used Solid Phase Micro-Extraction (SPME) headspace analysis to show that the natural odor neutralizers of the preferred embodiments of the present invention formed molecular complexes with the volatile sulfur compounds responsible for garlic odor and thus prevented those compounds from reaching the headspace. Accordingly, these natural odor neutralizers are well suited for breath mint and chewing gum applications and they provide total malodor management of endogenous bad breath due to the presence of gram-negative bacteria in the mouth and exogenous bad breath due to the ingestion of malodorous foods and drinks such as garlic, onion, and coffee.

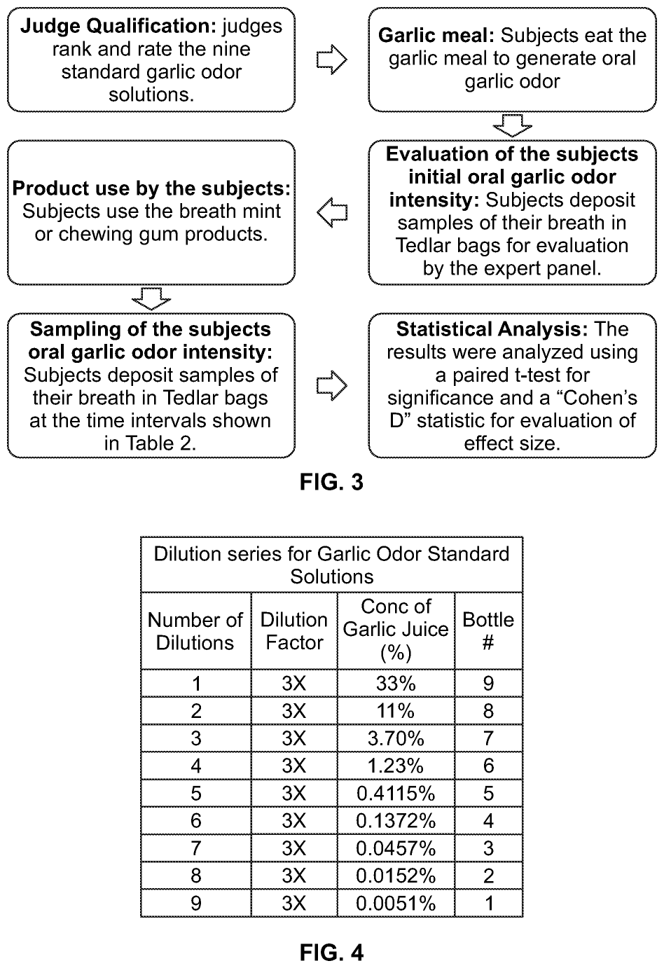

The protocol for the assessment is shown in FIG. 3.

MATERIALS AND METHODS

TEST SUBJECTS: Five subjects were used for the breath mint and chewing gum studies. The subjects utilized for the breath mint and chewing gum studies were drawn from a population of healthy Belle Aire Creations employees. All subjects were 18 years of age or older and were male. Five subjects were used for each study. All subjects were non-smokers, had good oral hygiene habits, and had no tooth decay or periodontal disease. The subjects were asked to refrain from consuming spicy food eight hours prior to the study.

EXPERT EVALUATORS: Five expert evaluators were used for the breath mint and chewing gum studies. The expert evaluators all had at least 15 years of experience smelling samples. Prior to each smelling session the evaluators were asked to rank nine garlic odor standard solutions from low to high intensity and to rate the odor intensities of the solutions on a 0-10 odor intensity scale. The garlic odor standard solutions used for this exercise are presented in FIG. 4.

PRODUCTS EVALUATED:

Breath mints study: Four (4) Belle Aire breath mints (0.70 g) containing 0.10% TMM® NANO with peppermint flavor were compared to the same product not containing TMM® NANO.

Chewing gum study: One (1) stick of Belle Aire slab-type chewing gum (2.9 g) containing 0.1% TMM® NANO was compared to the same product not containing TMM® NANO.
GARLIC STIMULUS FOR GENERATION OF ORAL GARLIC ODOR AND GARLIC ODOR STANDARD SOLUTIONS:
a) Garlic Valley Farms-brand cold pressed garlic juice was used both for production of oral garlic odor and for the garlic odor standard solutions. This product is available from Amazon.com: http: //www.amazon.com/Garlic-Juice-Valley-Farms-Bottles/dp/B003DQBLCO/ref=sr_1_1_a_it?ie=UTF8&qid=1458328657&sr=8-1&keywords=Garlic+juice
The garlic juice was refrigerated immediately upon receipt and kept refrigerated between uses.
b) Dorot-brand frozen crushed garlic cubes in combination with Garlic Valley Farms-brand cold pressed garlic juice was used to generate a robust oral garlic odor. Dorot-brand frozen crushed garlic is available at, for example, Trader Joe's, Whole Foods, and Albertsons.

GARLIC BREATH SAMPLING BAGS

The subjects' garlic breath was collected in a one-liter polyvinyl fluoride bag (Tedlar-brand) with a push/pull lock valve. These bags are available from Supelco, Inc., catalog number 24633.

GARLIC ODOR STANDARD SOLUTIONS

A set of nine, threefold dilutions of neat Garlic Valley Farms-brand cold pressed garlic juice was used to train and credential the judges. This dilution series is presented in FIG. 3.

QUALIFICATION OF THE JUDGES

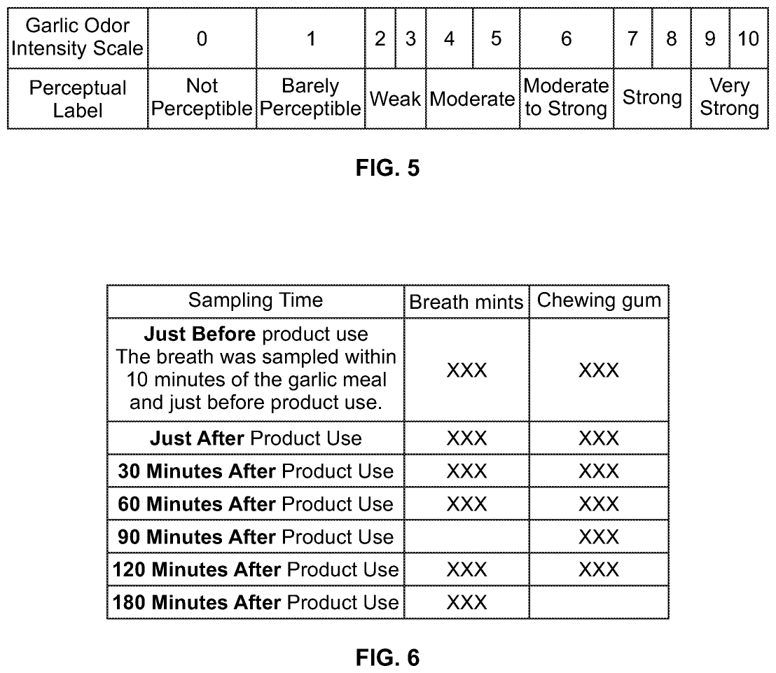

Each run was initiated with the evaluation of nine garlic odor standards. The judges were asked to rank the nine garlic odor standard solutions from lowest to highest odor intensity and to rate them using the 0-10 garlic odor intensity scale presented below in FIG. 5. This evaluation verified each judge's ability to smell garlic odor.

GARLIC MEAL: GENERATION OF ORAL GARLIC ODOR

The garlic meal was designed to produce strong instant and sustained oral garlic odor. The instant oral garlic odor was obtained by swishing neat, cold-pressed garlic juice in the mouth, and the sustained oral garlic odor was obtained by eating crushed raw garlic. Such a meal should cover virtually all types of garlic ingestion.

Two cubes of Dorot-brand frozen crushed garlic (4.3 g/cube) were brought up to room temperature (five seconds in the microwave) and spread onto a Nabisco-brand saltine cracker. The garlic/saltine cracker meal was chewed by the subject for one minute and swallowed, followed by rinsing and swallowing 25 mL of water. Approximately five minutes later, 25 mL of neat Garlic Valley Farms-brand cold pressed garlic juice was swished in the mouth for one minute and expectorated. This step was followed by 25 mL of water which was swished and swallowed. This procedure was found to reliably generate a very strong initial oral garlic odor on the 0-10 garlic odor intensity scale.
EVALUATION OF THE SUBJECT'S INITIAL (BASELINE) ORAL GARLIC ODOR INTENSITY

Following the garlic meal, each subject was instructed to breathe in gently through his nose and then to exhale slowly into the Tedlar-brand bag so as to draw the garlic odor from the oral cavity and deposit it into the bag. Generally, one breath was all that was necessary to inflate the bag. The bag was then sealed by utilizing its push/pull locking mechanism.

The intensity of the garlic odor captured in the Tedlar-brand bag was evaluated by the panel of five judges, usually within one hour after the completion of a run.

PRODUCT USE BY THE SUBJECTS

BREATH MINTS

The breath mint study consisted of two runs. Each subject was issued a small coded cup containing four breath mints (0.7 g). The cups were randomly assigned with some subjects receiving breath mints not containing TMM® NANO and some receiving breath mints with TMM® NANO. On the second run the subjects receiving the breath mints containing TMM® NANO received breath mints not containing TMM® NANO and vice versa.

The subjects were instructed to suck on the four breath mints and to allow them to dissolve in their mouths with no chewing. This step took approximately two minutes. The subjects were asked to swish the saliva generated by the breath mints before swallowing.

CHEWING GUM

Similar to the breath mint study, the chewing gum study consisted of two runs. Each subject was issued one stick (2.8 g) of slab-type chewing gum. The chewing gum sticks were randomly assigned with some subjects receiving chewing gum containing TMM® NANO and some receiving chewing gum not containing TMM® NANO. On the second run the order of product assignment was reversed. The subjects were instructed to chew the gum for four minutes and then to expectorate it.

SAMPLING OF THE SUBJECT'S ORAL GARLIC ODOR INTENSITY

At the appropriate time interval shown in FIG. 6, the subject was instructed to deposit a sample of his breath in the Tedlar-brand bag by breathing in slowly through his nose and then exhaling slowly into the Tedlar-brand bag.

The breath mint and chewing gum studies were sampled at slightly different time intervals. The breath mint study was sampled out to 180 minutes since at 120 minutes there was still a barely noticeable scent of garlic. The chewing gum study was sampled out to 120 minutes since at this point there was no detectable garlic odor. Since the garlic odor was totally gone at 120 minutes, a 90-minute sampling time was added to this run to better understand the dynamics of the garlic odor intensity decay.

Each of the five subjects produced a Tedlar-brand bag containing their breath sample for each of the time intervals comprising the study. Each of the Tedlar bags was evaluated by each of the five judges who rated the garlic odor intensity of the breath captured in the bag on the 0-10 garlic odor intensity scale illustrated in FIG. 5. In this way, each subject's breath was evaluated five times.

STATISTICAL ANALYSIS

A paired t-test was used to determine if the reduction of oral garlic odor was due to the addition of TMM® NANO to the formulation and not to something else.

A “Cohan's D” statistic, also referred to as “Effect Size,” was used to determine the probability that the TMM® NANO effect will be perceivable by the consumer. The Effect Size is the size of the effect represented in units of standard deviation. Mathematically it is represented as the following:

$$\begin{matrix}Effect \\ Size\end{matrix} = \frac{\begin{matrix}[ \begin{matrix}Average garlic odor intensity \\ for subjects using the \\ product w \text{/} o TMM \text{-} Nano\end{matrix} ] = \\ [ \begin{matrix}Average garlic odor intensity \\ for subjects using the \\ product w \text{/} TMM \text{-} Nano\end{matrix} ]\end{matrix}}{[ Average standard deviation for the two averages ]}$$

The probability that a consumer can differentiate between two products having a given effect size was obtained by consulting a table of effect size versus probability of detecting the difference.

SPME ANALYSIS OF THE MOLECULAR COMPONENTS OF GARLIC ODOR

In support of the sensory studies, Belle Aire scientists chemically analyzed the subjects' saliva for the components of garlic odor. The following saliva samples were analyzed:
Saliva collected after the subjects ate the garlic meal and just before they used the product (“Just Before” sampling period in FIG. 6).
Saliva collected after the subjects ate the garlic meal, and just after they used the breath mint product containing TMM® NANO. (“Just After” sampling period in FIG. 6).
Saliva from two subjects were analyzed with each saliva sample analyzed three times.

Using Solid Phrase Micro-Extraction (SPME) coupled with gas chromatography-mass spectroscopy (GC-MS), the saliva samples were analyzed for the presence of allyl methyl sulfide and diallyl disulfide), two major contributors to garlic odor:

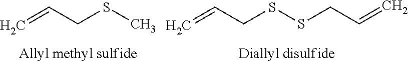

Five mL of the subject's saliva was transferred to a 10 mL SPME vial and the headspace above the saliva was sampled for one hour at 40® C.

GENERATION OF ORAL MALODOR BREATH MINT STUDY

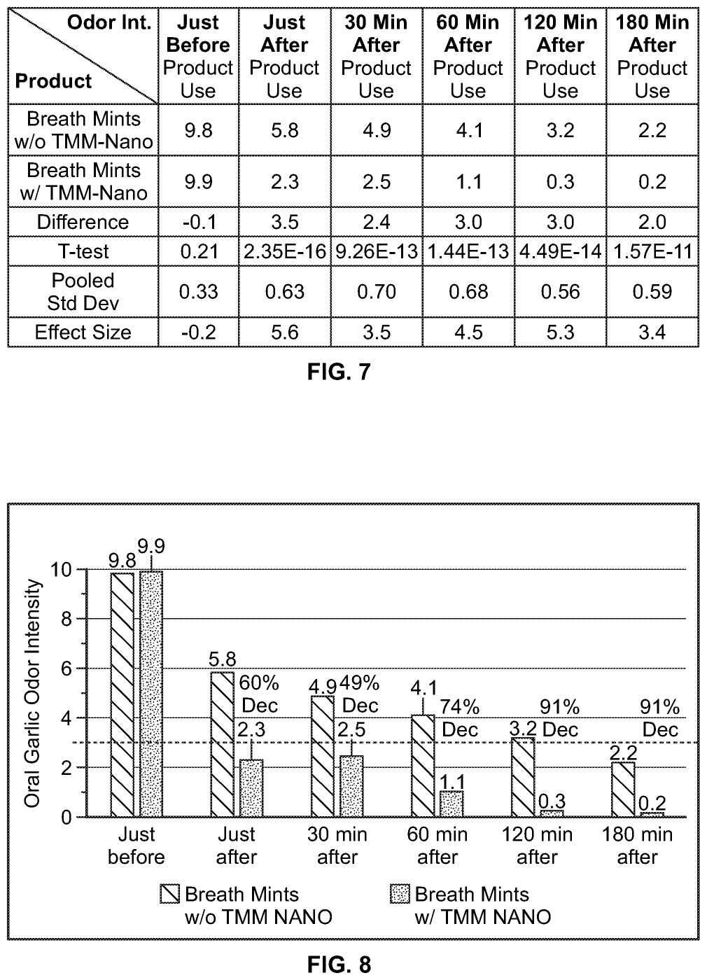

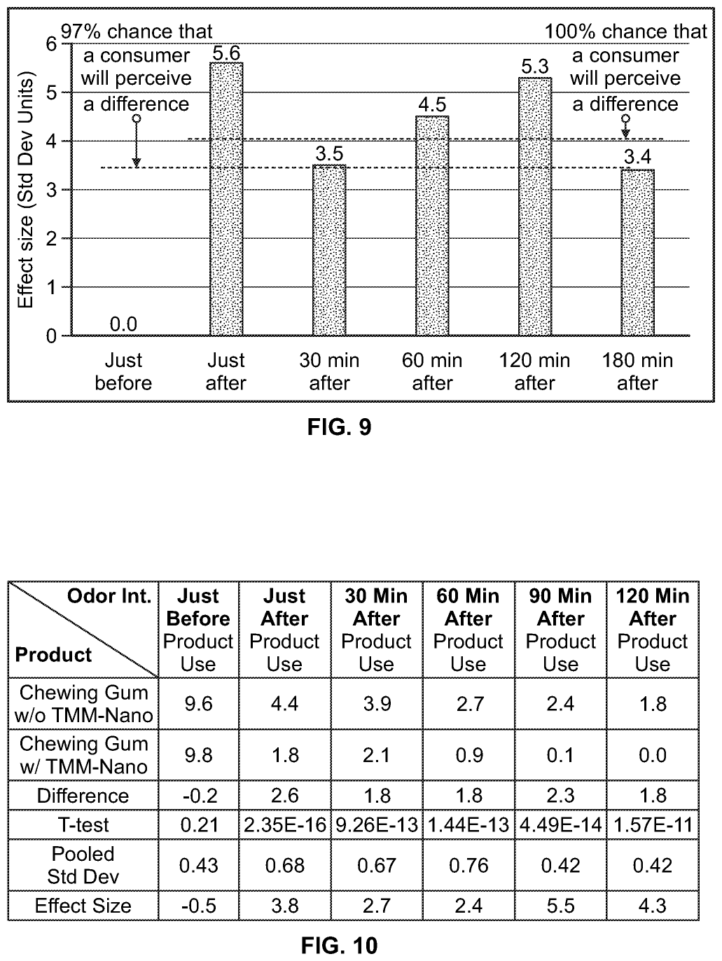

FIGS. 7, 8, and 9 summarize the results for the breath mints study. FIG. 7 presents: (a) the data used to construct FIGS. 8 and 9; (b) the results of the t-test analysis of the data; and (c) the effect size.

Note that in FIG. 8, the t-test results are extremely small. This detail is common with data where the effect of the treatment (in this case addition of TMM® NANO) is large.

FIG. 8 presents the oral garlic odor intensities observed by the five expert evaluators who smelled the subject's breath that had been collected in Tedlar-brand bags. The left side of the chart presents the garlic odor intensities based on the 0-10 category scale used by the judges and the right side of the chart presents the intensities based on the perceptual labels in the 0-10 scale. For example, a rating of 1 is a Barely Perceptible oral garlic odor whereas a rating of 9 is a Very Strong perceived oral garlic odor.

FIG. 8 reveals two interesting results. First, breath mints containing peppermint flavor alone do not reduce oral garlic odor below the unpleasant level. The expert evaluators observed that oral garlic odor intensities above Weak (3 on the 0-10 scale) were unpleasant. Second, addition of TMM® NANO brought the oral garlic odor intensity below Weak, maintained it at Weak at the 30-minute mark, and subsequently reduced it to Barely Perceptible and below.

FIG. 9 presents the Effect Size results. Effect Size, or Cohan's D statistic, is the size of the reduction of oral garlic odor intensity due to the addition of TMM® NANO to the product (signal) divided by the standard deviation associated with this reduction (noise). The statistical measures of difference such as the t-test are meaningless when it comes to judging whether a consumer will perceive the effect. In FIG. 9, the Effect Size that was calculated in FIG. 8 is presented graphically.

The two red dashed lines in FIG. 9 show the 97 percent chance and the 100 percent chance levels that the consumer will perceive the effect of the addition of TMM® NANO to the product. The judges reported that the differences in odor intensity due to addition of TMM® NANO were very obvious, so virtually all consumers using the product will perceive the effect.

CHEWING GUM STUDY

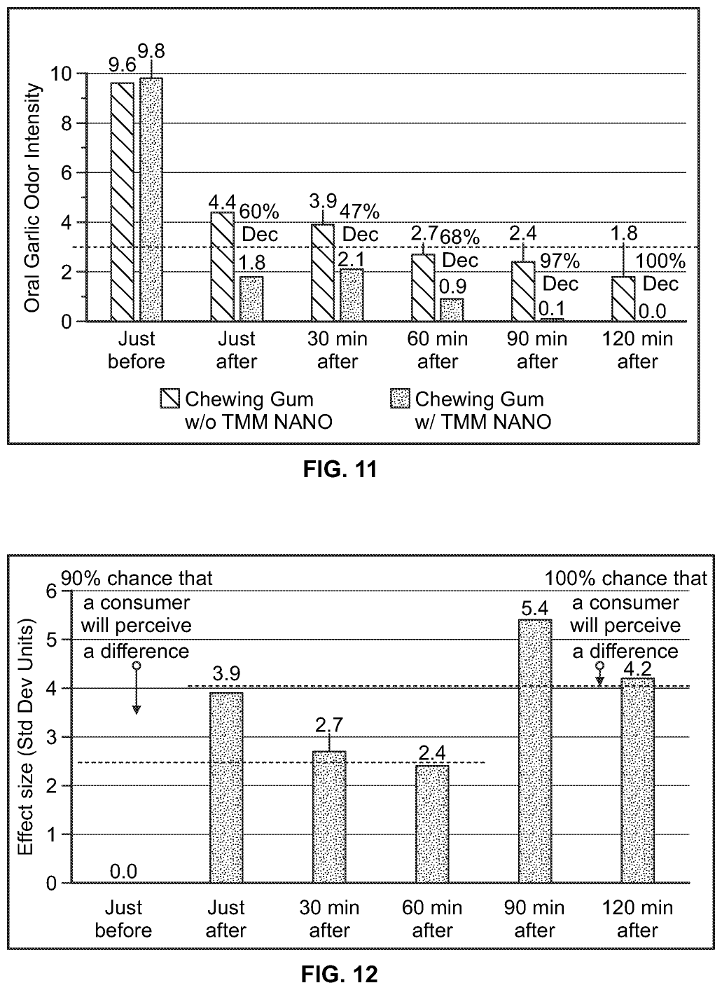

FIGS. 10, 11, and 12 summarize the results for the chewing gum study. FIG. 10, in addition to presenting the data used to construct FIGS. 11 and 12, presents the results of the statistical analysis of the data.

As was the case for the breath mints, FIG. 11 presents the oral garlic odor intensities observed by the five expert evaluators who smelled each subject's breath that had been collected in Tedlar-brand bags. The right side of the chart presents the garlic odor intensities expressed numerically by the judges who used a 0-10 category scale for the ratings. The right side of the chart presents the perceptual labels for the numerical ratings. Thus, a rating of 1 is Barely Perceptible perceived oral garlic odor, and a rating of 9 is a Very Strong perceived oral garlic odor.

Comparison of FIG. 11 to FIG. 8 shows that chewing gum is more effective than breath mints at reducing oral garlic odor intensity. This result is likely due to the mechanical action of chewing the gum, which generates a lot of saliva that washes out some of the garlic volatiles. The addition of TMM® NANO to the chewing gum brings the oral garlic odor intensity well under the Weak level, which is 3 on the 0-10 category scale used by the judges to rate the intensity. Chewing gum containing only peppermint flavor, on the other hand, does not have this effect. There is a residual garlic odor that is still present two hours after the use of the product.

FIG. 12 is the graphical presentation of the Effect Size that was calculated in FIG. 10. As described in FIG. 9, Effect Size is a signal-to-noise measurement that compares the size of the oral garlic odor reduction due to addition of TMM® NANO to the product to the data noise (standard deviation) associated with the reduction.

The two red dashed lines in FIG. 12 show the 90 percent chance and 100 percent chance levels that the consumer will perceive the effect of the addition of TMM® NANO the product. These two chance levels were obtained from a statistical table. The judges, however reported that the differences in odor intensity due to addition of TMM® NANO to the product were very obvious, so in practice virtually all consumers using the product will perceive the effect.

SPME ANALYSIS OF THE MOLECULAR COMPONENTS OF GARLIC ODOR

Saliva collected from two subjects was analyzed for the presence of allyl methyl sulfide and diallyl disulfide, the two major contributors to garlic odor.

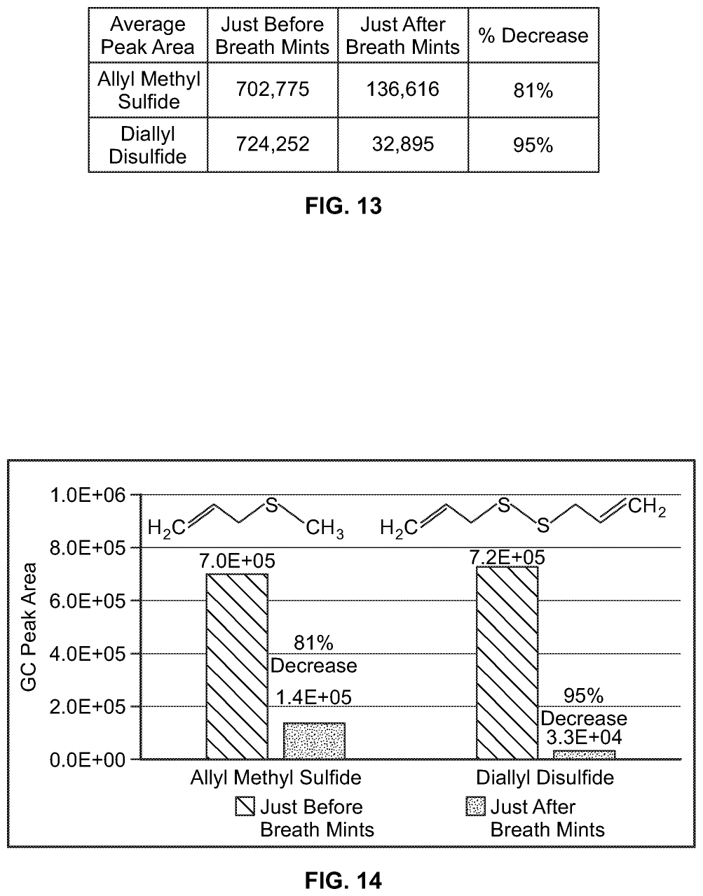

Saliva samples were collected just after the subjects had finished their garlic meal (Just Before Product Use in FIG. 6) and again just after the subjects had used the Breath Mint product containing TMM® NANO (Just After Product Use in FIG. 6). Each saliva sample was analyzed three times. The results of this study are presented in FIGS. 13 and 14.

The decrease in the concentrations of allyl methyl sulfide and diallyl disulfide in the headspace due to the presence of TMM® correlate nicely with the sensory results obtained for the breath mint study presented above in FIGS. 7, 8, and 9. Accordingly, there is an objective verification of the subjective results obtained from the sensory studies. These results demonstrate that TMM® is forming molecular complexes with the major volatile components responsible for garlic odor thus reducing their volatility to the point that they are no longer perceptible by the nose.

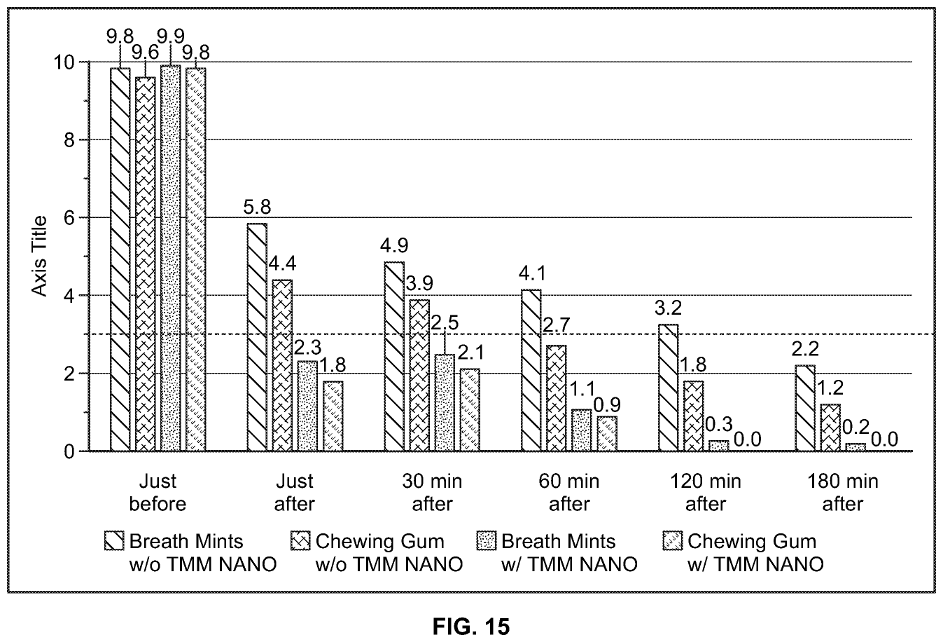

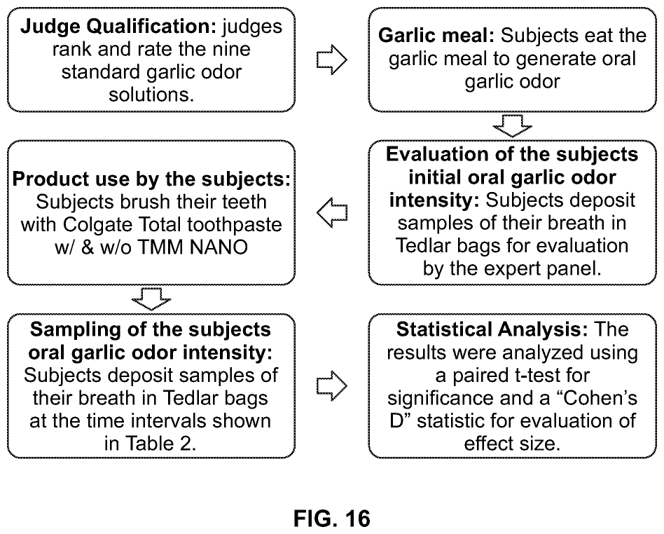

FIG. 15 summarizes the breath mint and chewing gum studies. In order to show both the immediate and sustained malodor counteraction ability of TMM® NANO, the garlic meal was designed to generate an immediate very strong garlic odor that persisted for at least three hours. FIG. 15 shows that addition of TMM® NANO brings the oral garlic odor down below Weak which is 3 on the 0-10 scale used by the judges to rate garlic odor intensity. At three or below, the judges found that the residual garlic odor was no longer unpleasant. The size of this reduction was found by Effect Size analysis to have a 90 percent chance or better that a consumer will perceive this difference. This level of chance was found by comparing the Effect Size to a statistical table relating effect size to chance of perception. The judges, however, found that the size of the effect was very perceptible, suggesting that in practice 100 percent of consumers will find that addition of TMM® NANO to either breath mints or chewing gum will very effectively reduce the intensity of their garlic breath to level that is not perceptible to someone else. Since the study was intentionally designed so that the garlic meal would generate an oral garlic odor that was very strong and persistent, any product that will freshen a breath contending this level of garlic odor will freshen up any type of breath odor.

TOOTHPASTE

The addition of TMNI® NANO to toothpaste at 0.1 percent effectively eliminates volatile sulfur compounds associated with halitosis. Belle Aire's sensory science group assessed oral garlic odor that had been induced in the oral cavity of the subjects by their ingesting crushed garlic followed by swishing and expectorating neat, cold-pressed garlic juice. The size of this oral garlic odor intensity reduction was such that any user of the product would perceive the effect.

Belle Aire's analytical team used Solid Phase Micro-Extraction (SPME) headspace analysis to show TMM® NANO exhibited its effect by forming molecular complexes with the volatile sulfur compounds responsible for garlic odor and thus preventing them from reaching the headspace.

TEST SUBJECTS

The five subjects used for the toothpaste study were drawn from a population of healthy Belle Aire Creations employees. All subjects were 18 years of age or older and were male. All subjects were non-smokers, had good oral hygiene habits, and had no tooth decay or periodontal disease. The subjects were asked to refrain from consuming spicy food eight hours prior to the study.

EXPERT EVALUATORS

Five expert evaluators were used for the toothpaste study. All of the expert evaluators had at least 15 years of experience smelling samples. Prior to each smelling session the evaluators were asked to rank nine garlic odor standard solutions from low to high intensity and to rate the odor intensities of the solutions on a 0-10 odor intensity scale. The garlic odor standard solutions used for this exercise are presented in FIG. 4.

PRODUCT EVALUATED

Colgate Total Advanced Whitening toothpaste (2.5 g) containing 0.1 percent TMM® NANO was compared to the same product not containing TMM® NANO.
GARLIC STIMULUS FOR GENERATION OF ORAL GARLIC ODOR AND GARLIC ODOR STANDARD SOLUTIONS
(a) Garlic Valley Farms-brand cold pressed garlic juice was used both for production of oral garlic odor and for the garlic odor standard solutions. Recently Garlic Valley Farms has discontinued this product. We replaced it with Reese® which is available from Amazon.com. The garlic juice was refrigerated immediately after opening and kept refrigerated between uses.
(b) Dorot-brand frozen crushed garlic cubes in combination with Garlic Valley Farms-brand garlic juice was used to generate a robust oral garlic odor. Dorot-brand frozen crushed garlic is available at Trader Joe's, Whole Foods, and Albertsons.

GARLIC BREATH SAMPLING BAGS

The subject's garlic breath was collected in a 1 L Tedlar-brand bag with a push/pull lock valve. These bags are available from Supelco, Inc., catalog number 24633.

GARLIC ODOR STANDARD SOLUTIONS

A set of nine, threefold dilutions, as shown in FIG. 4, of neat Garlic Valley Farms-brand garlic juice was used to train and credential the judges.

QUALIFICATION OF THE JUDGES

Each run was initiated with the evaluation of nine garlic odor standards. The judges were asked to rank the nine garlic odor standard solutions from lowest to highest odor intensity and to rate them using the 0-10 garlic odor intensity scale presented in FIG. 5. This evaluation verified each judge's ability to smell garlic odor.

GARLIC MEAL: GENERATION OF ORAL GARLIC ODOR

The garlic meal was designed to produce strong instant and sustained oral garlic odor. The instant oral garlic odor was obtained by swishing neat, garlic juice in the mouth, and the sustained oral garlic odor was obtained by eating crushed raw garlic.

Two cubes of Dorot-brand frozen crushed garlic (4.3 g/cube) were brought up to room temperature (five seconds in the microwave) and spread onto a Nabisco-brand saltine cracker.

The garlic/saltine cracker meal was chewed by the subject for one minute and swallowed, followed by rinsing and swallowing 25 mL of water. Approximately five minutes later, 25 mL of neat Garlic Valley Farms-brand garlic juice was swished in the mouth for one minute and expectorated. This step was followed by 25 mL of water which was swished and swallowed. This procedure was found to reliably generate a Very Strong initial oral garlic odor as measured by the 0-10 garlic odor intensity scale.
EVALUATION OF THE SUBJECT'S INITIAL (BASELINE) ORAL GARLIC ODOR INTENSITY

Following the garlic meal, the subject was instructed to breathe in gently through his nose and then to exhale slowly into the Tedlar-brand bag so as to draw the garlic odor from the oral cavity and deposit it into the bag. Generally, one breath was all that was necessary to inflate the bag. The bag was then sealed by utilizing its push/pull locking mechanism.

The intensity of the garlic odor captured in the Tedlar-brand bag was evaluated by the panel of five judges usually within one hour after the completion of a run.

PRODUCT USE BY THE SUBJECTS

This study consisted of two runs. Each subject was issued a toothbrush containing 2.5 g of toothpaste. The toothbrushes were randomly assigned with some subjects receiving toothpaste containing 0.1% TMM® NANO and some receiving toothpaste not containing TMM® NANO. The subjects brushed their teeth for one minute (15 seconds/quadrant) and then rinsed for 10 seconds with 25 mL of water. On the second run the subjects receiving the toothpaste containing TMM® NANO received toothpaste not containing TMM® NANO and vice versa.

SAMPLING OF THE SUBJECT'S ORAL GARLIC ODOR INTENSITY

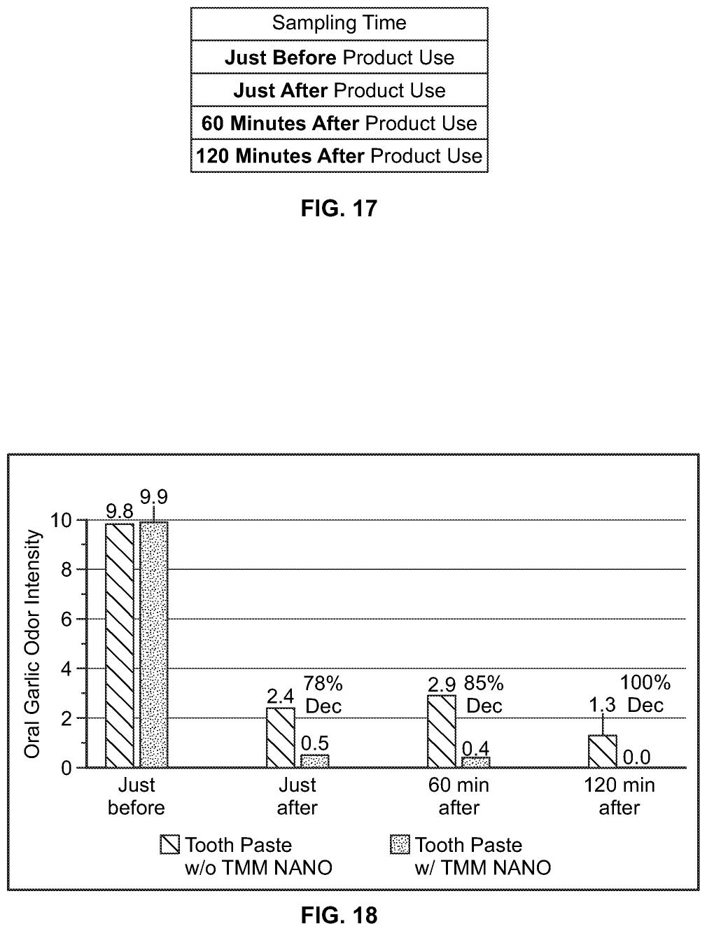

At the appropriate time interval, shown in FIG. 17, the subject was instructed to deposit a sample of his breath in the Tedlar-brand bag by breathing in slowly through his nose and then exhaling slowly through his mouth into the Tedlar bag.

Each of the five subjects produced a Tedlar-brand bag containing his or her breath sample for each of the time intervals comprising the study. Each of the Tedlar bags was evaluated by each of the five judges who rated the garlic odor intensity of the breath captured in the bag on the 0-10 garlic odor intensity scale illustrated in FIG. 5. In this way, each subject's breath was evaluated five times.

STATISTICAL ANALYSIS

A paired t-test was used to determine if the reduction of oral garlic odor was due to the addition of TMM® NANO to the formulation and not to something else. A “Cohan's D” statistic, or “Effect Size,” described above, was used to determine the probability that the TMM® NANO effect will be perceivable by the consumer.

The probability that a consumer can differentiate between two products having a given effect size was obtained by consulting a table of effect size versus probability of detecting the difference.

SPME ANALYSIS OF THE MOLECULAR COMPONENTS OF GARLIC ODOR

In support of the sensory studies, Belle Aire scientists chemically analyzed the subjects' saliva for the components of garlic odor. The following saliva samples were analyzed:
Saliva collected after the subjects ate the garlic meal and just before they used the product (“Just Before” sampling period in FIG. 18).
Saliva collected after the subjects ate the garlic meal, and just after they used the product containing TMM® NANO. (“Just After” sampling period in FIG. 18).
Saliva from two subjects were analyzed with each saliva sample analyzed three times.

Using Solid Phrase Micro-Extraction (SPME) coupled with gas chromatography-mass spectroscopy (GC-MS), the saliva samples were analyzed for the presence of allyl methyl sulfide and diallyl disulfide), two major contributors to garlic odor. Five mL of the subject's saliva was transferred to a 10 mL SPME vial and the headspace above the saliva was sampled for one hour at 40® C.

RESULTS AND DISCUSSION

In order to show both the immediate and sustained malodor counteraction ability of TMM® NANO, a garlic meal was designed that generated an immediate very strong garlic odor that persisted for at least three hours. This garlic meal consisted of eating crushed garlic followed by rinsing the mouth with cold pressed garlic juice.

TOOTHPASTE STUDY

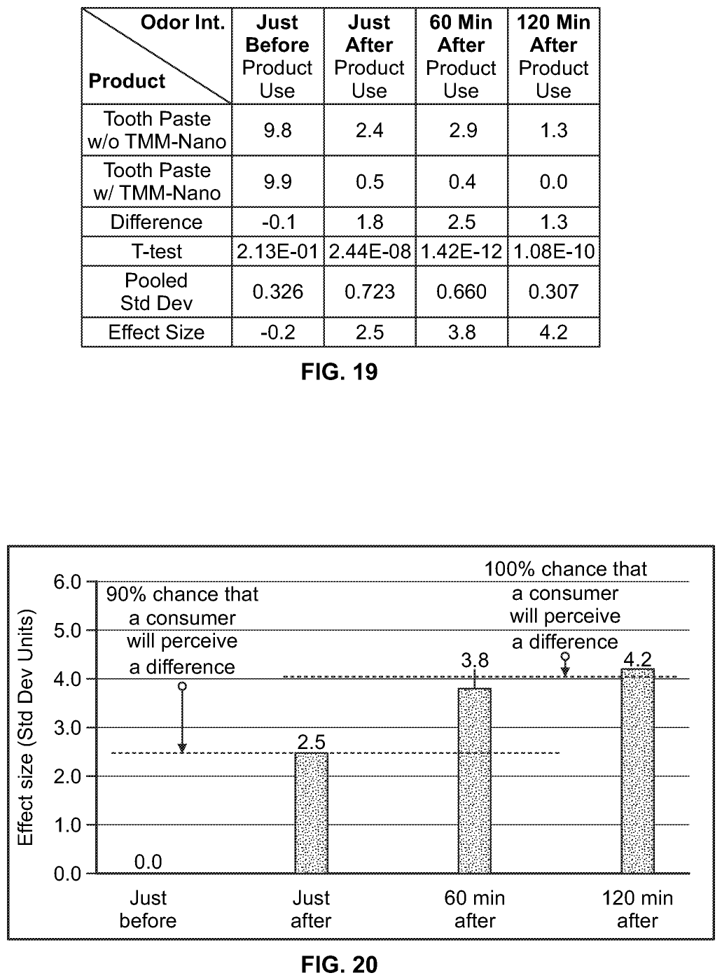

FIGS. 18 and 19 summarize the results for the toothpaste study. FIG. 19 presents the data used to construct FIG. 18 along with the results of the t-test analysis, and the effect size.

Note that in FIG. 19, the t-test results are extremely small. This result is common with data where the effect of the treatment (in this case addition of TMM® NANO) is large.

FIG. 18 presents the oral garlic odor intensities observed by the five expert evaluators who smelled the subject's breath that had been collected in Tedlar bags.

FIG. 18 reveals two interesting results. First, the mechanical action of toothbrushing coupled with the high intensity of its mint flavor reduces the oral garlic odor intensity down to a level that the judges rated as Weak relative to the just-before-toothbrushing garlic odor intensity. Secondly, the judges found that even at a Weak level the odor of garlic was quite noticeable on the subject's breath and was considered unpleasant.

FIG. 20 presents the Effect Size results. Effect Size, also referred to as the Cohan's D statistic, is the size of the reduction of oral garlic odor intensity due to the addition of TMM® NANO to the product (signal) divided by the standard deviation associated this reduction (noise). Statistical measures of difference such as the t-test are meaningless when it comes to judging whether a consumer will perceive the effect. In FIG. 20, the Effect Size that was calculated in FIG. 19 is presented graphically.

The two red dashed lines in FIG. 20 show the 90 percent and 100 percent chance levels that the consumer will perceive the effect of the addition of TMM® NANO to the product. The judges reported that the differences in odor intensity due to addition of TMM® NANO were obvious, obvious enough that at least 90 percent of the consumers using the product will notice the difference.

SPME ANALYSIS OF THE MOLECULAR COMPONENTS OF GARLIC ODOR

Saliva collected from two subjects was analyzed for the presence of allyl methyl sulfide and diallyl disulfide, the two major contributors to garlic odor.

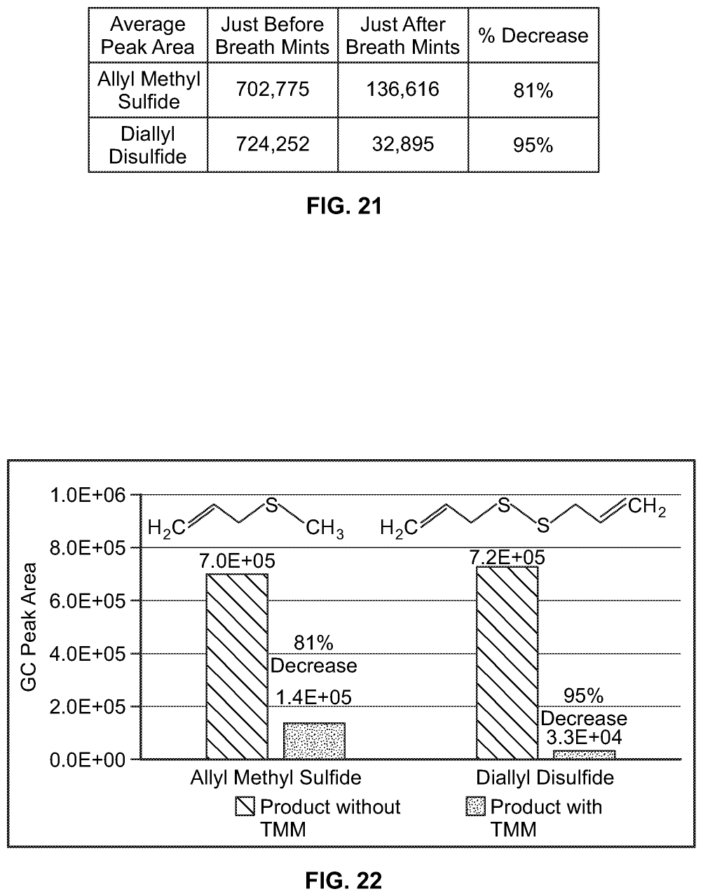

Saliva samples were collected just after the subjects had finished their garlic meal (Just Before Product Use in FIG. 17) and again just after the subjects had used the product containing TMM® NANO (Just After Product Use in FIG. 17). Each saliva sample was analyzed three times. The results of this study are presented below in in FIGS. 21 and 22.

The decrease in the concentrations of allyl methyl sulfide and diallyl disulfide in the headspace due to the presence of TMM® correlate nicely with the sensory results obtained for the breath mint study presented above in FIGS. 18, 19, and 20. Accordingly, there is an objective verification of the subjective results obtained from the sensory studies. These results demonstrate that TMM® is forming molecular complexes with the major volatile components responsible for garlic odor thus reducing their volatility to the point that they are no longer perceptible by the nose.

HOW TMM® WORKS

Two tools were used for the development of TMM® formulations: (a) computational chemistry utilizing quantum mechanics and mechanistic organic chemistry to both better understand the unique qualities malodor molecules, and to elucidate molecules that form molecular complexes with malodor molecules; and (b) use of Solid Phase Micro-Extraction (SPME) headspace analysis to confirm analytically that the molecules in the TMM® formulations were indeed forming molecular complexes with malodors thus preventing them from escaping into the headspace.

The computational chemistry and mechanistic organic chemistry studies suggested that three types of molecular complexes were feasible: (1) Covalent bond formation, (2) Magnetic-like forces of attraction, and (3) Mixed micelle formation.

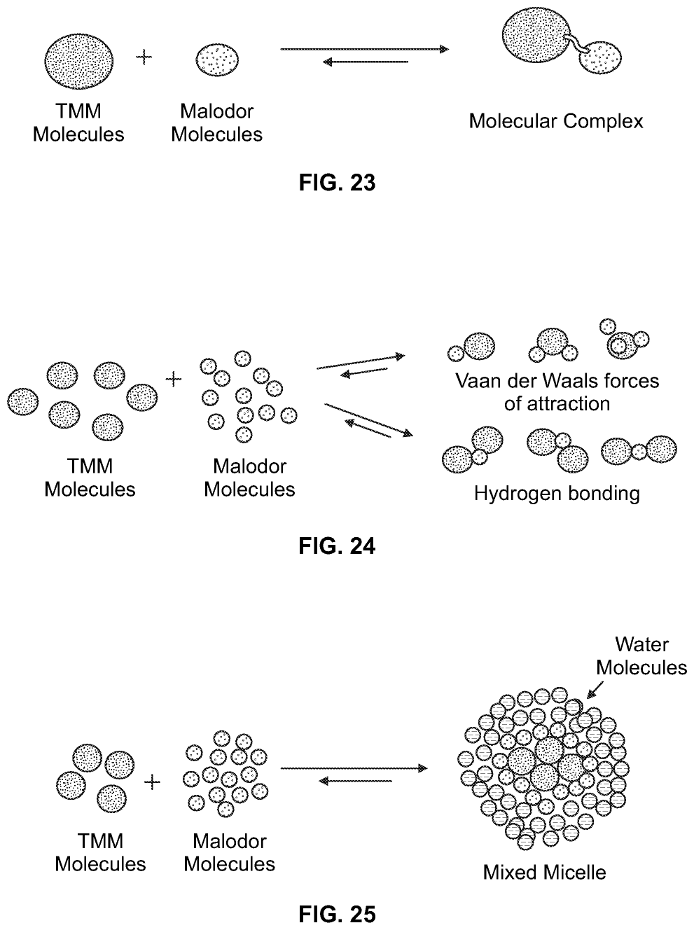

The malodor molecules 20 in a liquid, on a solid surface, or in the vapor phase will spontaneously, covalently bond to the appropriate TMM molecule 22, as shown in FIG. 23. The system is in dynamic equilibrium since these bonds are constantly being broken and reformed. The longer arrow 24 pointing to the right indicates that most malodor molecules 20 at any given instant in time are complexed to the TMM® molecule 22. TMM® molecules 22 having this capability were initially identified by computational chemistry techniques and subsequently confirmed by headspace analysis.

Malodor molecules 20 in a liquid, on a solid surface, or in the vapor phase will form molecular complexes held together by van der Waals forces of attraction and hydrogen bonding with the appropriate TMM® molecules 22, as shown in FIG. 24.

TMM® molecules 22 having the capability to form hydrogen bonds with malodor molecules 20 were also identified by computational chemistry techniques and verified by SPME headspace analysis.

The TMM® molecules 22 are water insoluble and will want to hide from the water molecules 26 by forming cluster 28, as shown in FIG. 25. The malodor molecules 20, which are more water-soluble but at the same time are attracted more to TMM® molecules 22 than to water molecules 26, will want to be between the TMM® molecules 22 and the water molecules 26 and will form a skin 30 around the cluster 28 of TMM® molecules 22. The water molecules 26 that surround skin 30 form mixed micelle 32.

The TMM® molecules 22 provide a multidimensional approach to malodor management by having the capability of complexing to malodor molecules present on a surface, in a liquid, or in the headspace above the surface. The molecular complexes formed are not volatile, thus the malodor molecules cannot diffuse into the atmosphere and be smelled.

While preferred embodiments of the present invention are shown and described, it is envisioned that those skilled in the art may devise various modifications of the present invention without departing from the spirit and scope of the appended claims.

I claim:

1. A time-release product for malodor reduction, the time-release product comprising an adsorbate and an adsorbent, the adsorbent comprising a zinc oxide nanoparticle and the adsorbate comprising a natural odor neutralizer.

2. The combination comprising the time release product of claim 1 and one of an oral care product and a personal care product.

3. The combination of claim 2 comprising an oral care product and the oral care product comprises at least one of a mouthwash, a mouth rinse, a toothpaste, a mint, a lozenge, a wafer, and a chewing gum.

4. The time-release product of claim 1, wherein the adsorbate comprises about 15 percent by weight of the time-release product and adsorbent comprises about 85 percent by weight of the time-release product.

5. The combination comprising the time release product of claim 4 and one of an oral care product and a personal care product.

6. The combination of claim 5 comprising an oral care product and the oral care product comprises at least one of a mouthwash, a mouth rinse, a toothpaste, a mint, a lozenge, a wafer, and a chewing gum.

7. A time-release product for malodor reduction, the time-release product comprising an adsorbate and an adsorbent, the adsorbent comprising a zinc oxide nanoparticle and the adsorbate comprising a natural odor neutralizer, the natural odor neutralizer comprising at least one of balsam copaiba, bees wax absolute, cardamon essence, hydroxycitronellal, ionone alpha, ionone beta, carvacrol, jasmine absolute, lavender absolute, mimosa absolute, neroli absolute, orange oil, orris absolute, osmanthus absolute, vanilla absolute, violet leaves absolute, davana oil, dimethyl anthranilate, elderflower oil, raspberry ketone, and natural ethyl alcohol.

8. The combination comprising the time release product of claim 7 and one of an oral care product and a personal care product.

9. The combination of claim 8 comprising an oral care product and the oral care product comprises at least one of a mouthwash, a mouth rinse, a toothpaste, a mint, a lozenge, a wafer, and a chewing gum.

10. The time-release product of claim 7, wherein the natural odor neutralizer comprises at least one of the following ingredients in the following weight percentages:

| | |
|---|---|
| balsam copaiba | 0.00020%-5.00000%; |
| bees wax absolute | 0.00001%-5.00000%; |
| cardamon essence | 0.00001%-5.00000%; |
| hydroxycitronellal | 0.05000%-8.00000%; |
| ionone alpha | 0.05000%-8.00000%; |
| ionone beta | 0.05000%-7.00000%; |
| carvacrol | 0.05000%-5.00000%; |
| jasmine absolute | 0.00200%-1.00000%; |
| lavender absolute | 0.00020%-1.00000%; |
| mimosa absolute | 0.00001%-1.00000%; |
| neroli absolute | 0.00001%-1.00000%; |
| orange oil | 0.00001%-5.00000%; |
| orris absolute | 0.00001%-0.30000%; |
| osmanthus absolute | 0.00001%-0.30000%; |
| vanilla absolute | 0.00001%-1.00000%; |
| violet leaves absolute | 0.00001%-0.40000%; |
| davana oil | 0.00001%-1.00000%; |
| dimethyl anthranilate | 0.00001%-2.00000%; |
| elderflower oil | 0.00001%-0.45000%; |
| raspberry ketone | 0.00001%-5.00000%; and |
| natural ethyl alcohol | 0.25%-6% |

11. The combination comprising the time release product of claim 10 and one of an oral care product and a personal care product.

12. The combination of claim 12 comprising an oral care product and the oral care product comprises at least one of a mouthwash, a mouth rinse, a toothpaste, a mint, a lozenge, a wafer, and a chewing gum.

13. A time-release product for malodor reduction, the time-release product comprising an adsorbate and an adsorbent, the adsorbent comprising a zinc oxide nanoparticle and the adsorbate comprising a natural odor neutralizer, the natural odor neutralizer comprising balsam copaiba, bees wax absolute, cardamon essence, hydroxycitronellal, ionone alpha, ionone beta, carvacrol, jasmine absolute, lavender absolute, mimosa absolute, neroli absolute, orange oil, orris absolute, osmanthus absolute, vanilla absolute, violet leaves absolute, davana oil, dimethyl anthranilate, elderflower oil, raspberry ketone, and natural ethyl alcohol.

14. The combination comprising the time release product of claim 13 and one of an oral care product and a personal care product.

15. The combination of claim 14 comprising an oral care product and the oral care product comprises at least one of a mouthwash, a mouth rinse, a toothpaste, a mint, a lozenge, a wafer, and a chewing gum.

16. The time-release product of claim 13, wherein the natural odor neutralizer comprises the following ingredients in the following weight percentages:

| | |
|---|---|
| balsam copaiba | 0.00020%-5.00000%; |
| bees wax absolute | 0.00001%-5.00000%; |
| cardamon essence | 0.00001%-5.00000%; |
| hydroxycitronellal | 0.05000%-8.00000%; |
| ionone alpha | 0.05000%-8.00000%; |
| ionone beta | 0.05000%-7.00000%; |
| carvacrol | 0.05000%-5.00000%; |
| jasmine absolute | 0.00200%-1.00000%; |
| lavender absolute | 0.00020%-1.00000%; |
| mimosa absolute | 0.00001%-1.00000%; |
| neroli absolute | 0.00001%-1.00000%; |
| orange oil | 0.00001%-5.00000%; |
| orris absolute | 0.00001%-0.30000%; |
| osmanthus absolute | 0.00001%-0.30000%; |
| vanilla absolute | 0.00001%-1.00000%; |
| violet leaves absolute | 0.00001%-0.40000%; |
| davana oil | 0.00001%-1.00000%; |
| dimethyl anthranilate | 0.00001%-2.00000%; |
| elderflower oil | 0.00001%-0.45000%; |
| raspberry ketone | 0.00001%-5.00000%; and |
| natural ethyl alcohol | 0.25%-6% |

17. The combination comprising the time release product of claim 16 and one of an oral care product and a personal care product.

18. The combination of claim 17 comprising an oral care product and the oral care product comprises at least one of a mouthwash, a mouth rinse, a toothpaste, a mint, a lozenge, a wafer, and a chewing gum.
